(19) United States
(12) Patent Application Publication
(10) Pub. No.: US 2022/0175720 A1
(43) Pub. Date: Jun. 9, 2022

(54) COMBINED PREPARATION COMPRISING AN ANTHOCYANIN COMPOSITION AND AN ANTIVIRAL AGENT

(71) Applicant: Evonik Operations GmbH, Essen (DE)

(72) Inventors: Jean-Luc HERBEAUX, Cascais (PT); Norbert WINDHAB, Hofheim (DE); Christoph BRÜCHER, Eschborn (DE); Anne BENEDIKT, Frankfurt (DE); Andrea ENGEL, Birmingham, AL (US); Maria STEINKE, Hoechberg (DE); Jochen BODEM, Geroldshausen (DE)

(73) Assignee: Evonik Operations GmbH, Essen (DE)

(21) Appl. No.: 17/598,587

(22) PCT Filed: Mar. 27, 2020
(86) PCT No.: PCT/EP2020/058662

§ 371 Date: Sep. 27, 2021

(30) Foreign Application Priority Data
Mar. 29, 2019 (EP) 19166083.6

(51) Int. Cl. A61K 31/352; A61K 36/45; A61K 36/87; A61K 31/7048; A61K 45/06; A61P 31/22; A61P 37/02

(52) U.S. Cl. CPC A61K 31/352; A61K 36/45; A61K 36/87; A61P 37/02; A61K 45/06; A61P 31/22; A61K 31/7048

(57) ABSTRACT

Combined preparations including an anthocyanin composition and an antiviral agent for use in treating or preventing a virus infection in a subject, wherein the anthocyanin composition includes an extract of black currants, an extract of bilberries, an extract of red grapes, and/or delphinidin 3 glucoside, wherein the virus is from the Herpesviridae family and the antiviral agent is a Herpesviridae antiviral agent, and wherein the anthocyanin composition and the antiviral agent are for simultaneous, separate or sequential use.

The present invention is related to combined preparations comprising an anthocyanin composition and an antiviral agent for use in treating or preventing a virus infection in a subject, wherein the anthocyanin composition comprises one or more of an extract of black currants, an extract of bilberries, an extract of red grapes, and/or delphinidin 3 glucoside, wherein the virus is from the Herpesviridae family and the antiviral agent is a Herpesviridae antiviral agent, and wherein the anthocyanin composition and the antiviral agent are for simultaneous, separate or sequential use.

Anthocyanins are water-soluble vacuolar pigments that may appear red, purple or blue, depending on the surrounding pH-value. Anthocyanins belong to the class of flavonoids, which are synthesized via the phenylpropanoid pathway. They occur in all tissues of higher plants, mostly in flowers and fruits and are derived from anthocyanidins by addition of sugars. Anthocyanins are glycosides of flavylium salts. Each anthocyanin thus comprises three component parts: the hydroxylated core (the aglycone); the saccharide unit; and the counterion. Anthocyanins are naturally occurring pigments present in many flowers and fruit and individual anthocyanins are available commercially as the chloride salts, e.g. from Polyphenols Laboratories AS, Sandnes, Norway. The most frequently occurring anthocyanins in nature are the glycosides of cyanidin, delphinidin, malvidin, pelargonidin, peonidin and petunidin.

It is known that anthocyanins, especially resulting from fruit intake, have a wide range of biological activities, including antioxidant, anti-inflammatory, antimicrobial and anti-carcinogenic activities, improvement of vision, induction of apoptosis, and neuroprotective effects. Particularly suitable fruit sources for the anthocyanins are cherries, bilberries, blueberries, black currants, red currants, grapes, cranberries, strawberries, and apples and vegetables such as red cabbage. Bilberries, in particular Vaccinium myrtillus, and black currants, in particular Ribes nigrum, are especially suitable.

Bilberries contain diverse anthocyanins, including delphinidin and cyanidin glycosides and include several closely related species of the genus Vaccinium, including Vaccinium myrtillus (bilberry), Vaccinium uliginosum (bog bilberry, bog blueberry, bog whortleberry, bog huckleberry, northern bilberry, ground hurts), Vaccinium caespitosum (dwarf bilberry), Vaccinium deliciosum (Cascade bilberry), Vaccinium membranaceum (mountain bilberry, black mountain huckleberry, black huckleberry, twin-leaved huckleberry), Vaccinium ovalifolium (oval-leafed blueberry, oval-leaved bilberry, mountain blueberry, high-bush blueberry).

Dry bilberry fruits of V. myrtillus contain up to 10% of catechin-type tannins, proanthocyanidins, and anthocyanins. The anthocyanins are mainly glucosides, galactosides, or arabinosides of delphinidin, cyanidin, and—to a lesser extent—malvidin, peonidin, and petunidin (cyanidin-3-O-glucoside (C3G), delphinidin-3-O-glucoside (D3G), malvidin-3-O-glucoside (M3G), peonidin-3-O-glucoside and petunidin-3-O-glucoside). Flavonols include quercetin- and kaempferol-glucosides.

The fruits also contain other phenolic compounds (e.g., chlorogenic acid, caffeic acid, o-, m-, and p-coumaric acids, and ferulic acid), citric and malic acids, and volatile compounds.

Black currant fruits (R. nigrum) contain high levels of polyphenols, especially anthocyanins, phenolic acid derivatives (both hydroxybenzoic and hydroxycinnamic acids), flavonols (glycosides of myricetin, quercetin, kaempferol, and isorhamnetin), and proanthocyanidins (between 120 and 166 mg/100 g fresh berries). The main anthocyanins are delphinidin-3-O-rutinoside (D3R) and cyanidin-3-O-rutinoside (C3R), but delphinidin- and cyanidin-3-O-glucoside are also found (Gafner, Bilberry—Laboratory Guidance Document 2015, Botanical Adulterants Program).

EP 1443948 A1 relates to a process for preparing a nutritional supplement (nutraceutical) comprising a mixture of anthocyanins from an extract of black currants and bilberries. Anthocyanins were extracted from cakes of fruit skin produced as the waste product in fruit juice pressing from V. myrtillus and R. nigrum. It could be shown that the beneficial effects of individual anthocyanins are enhanced if instead of an individual anthocyanin, a combination of different anthocyanins is administered orally, in particular a combination comprising both mono and disaccharide anthocyanins. It is thought that the synergistic effect arises at least in part from the different solubilities and different uptake profiles of the different anthocyanins.

Herpesviridae is a large family of DNA viruses that cause infections and certain diseases in humans such as oral herpes, chicken pox and infectious mononucleosis-like syndrome. Additionally, they can be connected to serious pathophysiology including Alzheimer's disease, Burkitt's lymphoma and Kaposi's sarcoma. Latent, recurring infections are also typical of this group of viruses, e.g. over 50% of the population worldwide is seropositive for human cytomegalovirus (hCMV). This ubiquitous herpes virus is the cause of widespread infections in humans and, although benign in immunocompetent hosts, patients with immature or compromised immune systems (as AIDS patients or organ transplant recipients) suffer from life-threatening complications.

In total more than 130 herpesviruses are known, however nine herpesvirus types are known to cause disease in humans, such as herpes simplex viruses 1 and 2 (HSV-1 and HSV-2, also known as HHV1 and HHV2) causing oral and/or genital herpes, as well as other herpes simplex infections, targeting mucoepithelial cells and neuronal latency. The varicella-zoster virus (VZV, HHV-3) is also targeting mucoepithelial cells (neuronal latency) and causes chickenpox and shingles. Epstein-Barr virus (EBV, HHV-4) is targeting B cells (including latency in B cells) and epithelial cells and is the cause of Infectious mononucleosis, Burkitt's lymphoma, CNS lymphoma in AIDS patients, post-transplant lymphoproliferative syndrome (PTLD), nasopharyngeal carcinoma and HIV-associated hairy leukoplakia. The human cytomegalovirus (HCMV, HHV-5) is targeting monocytes and epithelial cells (monocytes as site of latency) and causes infectious mononucleosis-like syndrome and retinitis. Human herpesvirus 6A and 6B (HHV-6A and HHV-6B) targets T cells (including site of latency) and causes sixth disease (Roseola infantum or Exanthem subitum). Human herpesvirus 7 (HHV-7) targets T cells as well and is the cause of drug-induced hypersensitivity syndrome, encephalopathy, hemiconvulsion-hemiplegia-epilepsy syndrome, hepatitis infection, post infectious myeloradiculoneuropathy, Pityriasis rosea, and the reactivation of HHV-4, leading to “mononucleosis-like illness”. The Kaposi's sarcoma-associated herpesvirus (KSHV, HHV-8) is targeting lymphocytes and other cells and causes Kaposi's sarcoma, primary effusion lymphoma, some types of multicentric Castleman's disease.

Herpesviruses are known for their ability to establish lifelong infections in the host, which is achieved through immune evasion. Interestingly, herpesviruses have many different ways of evading the immune system, such as mimicking human interleukin 10 (hIL-10) or downregulation of the major histocompatibility complex II (MHC II) in infected cells.

During the past decade a better understanding of the replication and disease-causing state of herpes viruses has been achieved in part due to the development of potent antiviral compounds that target these viruses. While some of these antiviral therapies are considered safe and efficacious (acyclovir, penciclovir), some have toxicities associated with them (ganciclovir and foscarnet). The most serious side effect of acyclovir is neurotoxicity, which usually occurs in subjects with compromised renal function who attain high serum concentrations of drug (Revankar et al., 1995). Neurotoxicity is manifest as lethargy, confusion, hallucinations, tremors, myoclonus, seizures, extrapyramidal signs, and changes in state of consciousness, developing within the first few days of initiating therapy. These signs and symptoms usually resolve spontaneously within several days of discontinuing acyclovir. Resistance of HSV to acyclovir has become an important clinical problem, especially among immunocompromised patients exposed to long-term therapy (Englund et al., 1990).

In the context it was surprisingly found, that an extract of black currants and bilberries in combination with an antiviral agent, mediates strong inhibition of herpes virus infection and replication, and there is a surprising synergistic effect. Thus, the present invention is based on the use of this combination of active agents in the treatment and prophylaxis of herpes infection. Therefore, this combination could be an important solution for a variety of herpes infections as well as their related diseases by combining the antiviral effect with its positive influence on cell viability and no toxicity.

The present invention is related to a combined preparation comprising an anthocyanin composition and an antiviral agent for use in treating or preventing a virus infection in a subject, wherein the anthocyanin composition comprises one or more of an extract of black currants, an extract of bilberries, an extract of red grapes, and/or delphinidin 3 glucoside, wherein the virus is from the Herpesviridae family and the antiviral agent is a Herpesviridae antiviral agent, and wherein the anthocyanin composition and the antiviral agent are for simultaneous, separate or sequential use.

It is preferred, when the anthocyanin composition comprises an extract of blackcurrants and bilberries. Moreover, it is preferred, when the combined preparation is a composition comprising the anthocyanin composition and the antiviral agent.

The invention also refers to an anthocyanin composition comprising one or more of an extract of black currants, an extract of bilberries, an extract of red grapes, and/or delphinidin 3 glucoside for use in treating or preventing a virus infection in a subject, wherein the virus is from the Herpesviridae family and the antiviral agent is a Herpesviridae antiviral agent, and wherein the composition is to be administered in combination with an antiviral agent active against the virus, or wherein the composition is to be administered to a subject treated with the antiviral agent.

The invention also relates to an anti-viral agent for use in treating or preventing a virus infection in a subject, wherein the virus is from the Herpesviridae family and the antiviral agent is a Herpesviridae antiviral agent, and wherein the antiviral agent is to be administered: (i) in combination with an anthocyanin composition comprising one or more of an extract of blackcurrants, an extract of bilberries, an extract of red grapes, or delphinidin 3 glucoside active against the virus; or (ii) to a subject treated with the anthocyanin composition.

As used herein the term “Herpesviridae antiviral agent” refers to an agent that can be used to treat or prevent an infection by a virus from the Herpesviridae family, and can itself be active against the virus or can be a prodrug that is metabolized in the body to an active agent. An example of the latter is valganciclovir, which is a prodrug of ganciclovir.

As used herein a combined preparation is one which comprises separately packaged active components which are to be combined in use, i.e. by being administered simultaneously, separately or sequentially to the subject.

It is preferred, when the antiviral agent is an inhibitor of DNA replication, optionally wherein the antiviral agent is a DNA polymerase inhibitor or a DNA terminase complex inhibitor. In particular, the DNA polymerase inhibitor may be a nucleoside analogue or a pyrophosphate analogue. It is further preferred, when the antiviral agent is one or more of acyclovir, ganciclovir, valganciclovir, foscarnet, famciclovir, penciclovir, or valaciclovir, or letermovir, preferably wherein the antiviral agent is acyclovir.

In one embodiment the combined preparation, composition or antiviral agent is for use in treating or preventing a virus infection, wherein the virus is from the sub-family Alphaherpesvirinae, preferably wherein the subject is human.

In another embodiment the combined preparation, composition or antiviral agent according to the present invention is especially for use in treating or preventing a virus infection in a human host is selected from
herpes simplex viruses 1 and 2 (HSV-1 and HSV-2, HHV1 and HHV2),
varicella-zoster virus (VZV, HHV-3),
Epstein-Barr virus (EBV, HHV-4),
human cytomegalovirus (HCMV, HHV-5),
human herpesvirus 6A and 6B (HHV-6A and HHV-6B),
human herpesvirus 7 (HHV-7), and
Kaposi's sarcoma-associated herpesvirus (KSHV, HHV-8).

The virus is preferably HSV-1 and the composition preferably suppresses viral infection.

The combined preparation, composition or antiviral agent for use is especially suitable when: (i) the virus is CMV and the antiviral agent is ganciclovir; or (ii) the virus is HSV-1 or EBV and the antiviral agent is acyclovir.

Moreover, herpesviruses represent the most frequently detected pathogens in the brain. Under constant immune pressure, these infections are largely asymptomatic in healthy hosts. However, many neurotropic herpesviruses have been directly connected with central nervous system pathology in the context of other stressors and genetic risk factors. There are indications that neurotropic herpesviruses, such as herpes simplex virus 1 (HSV-1) and human herpesvirus 6 (HHV-6) contribute to neurodegenerative disease pathology, such as Alzheimer's disease (AD) (Hogestyn et al., Neural Regeneration Research 13 (2), 211-221, 2018). For example, the herpes simplex virus HSV-1 has been found in the same areas as amyloid plaques. It has been shown that HSV-1 induces AD-related pathophysiology and pathology, including neuronal production and accumulation of amyloid beta (A13), hyperphosphorylation of tau proteins, dysregulation of calcium homeostasis, and impaired autophagy (Harris & Harris Frontiers in Aging Neuroscience Vol 10 (48), 2018). This suggested the possibility that AD could be treated or prevented with antiviral medication.

It is further also preferred to use the combined preparation, composition or antiviral agent according to the present invention for treating or preventing a virus infection with Ateline herpesvirus 1 (spider monkey herpesvirus), Bovine herpesvirus 2 (which causes bovine mammillitis and pseudo-lumpyskin disease), Cercopithecine herpesvirus 1 (also known as Herpes B virus, causes a herpes simplex-like disease in macaques, usually fatal if symptomatic and untreated in humans), Macacine herpesvirus 1,

Bovine herpesvirus 1 (causes infectious bovine rhinotracheitis, vaginitis, balanoposthitis, and abortion in cattle), Bovine herpesvirus 5 (causes encephalitis in cattle), Bubaline herpesvirus 1, Caprine herpesvirus 1 (causes conjunctivitis and respiratory disease in goats), Canine herpesvirus 1 (causes a severe hemorrhagic disease in puppies), Equine herpesvirus 1 (causes respiratory disease, neurological disease/paralysis, and spontaneous abortion in horses), Equine herpesvirus 3 (causes coital exanthema in horses), Equine herpesvirus 4 (causes rhinopneumonitis in horses), Equine herpesvirus 8, Equine herpesvirus 9, Feline herpesvirus 1 (causes feline viral rhinotracheitis and keratitis in cats), Suid herpesvirus 1 (causes Aujeszky's disease, also called pseudorabies),

Anatid herpesvirus 1, Columbiform herpesvirus 1, Gallid herpesvirus 2 (causes Marek's disease), Gallid herpesvirus 3 (GaHV-3 or MDV-2), Meleagrid herpesvirus 1 (HVT), Peacock herpesvirus 1 Gallid herpesvirus 1 (causes infectious laryngotracheitis in birds), Psittacid herpesvirus 1 (causes Pacheco's disease in birds),

Porcine herpesvirus 2 (causes inclusion body rhinitis in swine),

Alcelaphine herpesvirus 1 (causes bovine malignant catarrhal fever), Alcelaphine herpesvirus 2 (causes an antelope and hartebeest version of MCF), Ateline herpesvirus 2, Bovine herpesvirus 4, Cercopithecine herpesvirus 17, Equine herpesvirus 2 (causes equine cytomegalovirus infection), Equine herpesvirus 5, Equine herpesvirus 7, Japanese macaque rhadinovirus, Leporid herpesvirus 1, Murid herpesvirus 4 (Murine gamma herpesvirus-68, MHV-68),

Cyprinid herpesviruses 1, 2 and 3 (CyHV1, CyHV2 and CyHV3) causing disease in common carp, goldfish and koi respectively.

As noted above, the anthocyanin composition comprises one or more of an extract of black currants, an extract of bilberries, an extract of red grapes, and/or delphinidin 3 glucoside.

In a preferred embodiment, the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and/or bilberries and/or red grapes.

It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%.

It is preferred, according to the present invention, when the extract is an alcoholic extract, preferably a methanol extract. The extract is preferably produced by a process comprising the steps of
extraction of black currants and/or bilberries,
purification via chromatography,
mixing of the extract(s) with water and
spray-drying of the mixture.

One example of such a process is disclosed in EP1443948.

In a preferred embodiment, maltodextrin is added to the composition.

The composition according to the present invention preferably contains at least three monosaccharide anthocyanins. Moreover, it preferably contains at least one monosaccharide anthocyanin in which the saccharide is arabinose or at least one disaccharide anthocyanin in which the disaccharide is rutinose. The composition preferably contains anthocyanins with at least two different aglycones, more preferably at least four. Especially preferably the composition contains anthocyanins in which the aglycone units are cyanidin, peonidin, delphinidin, petunidin, malvidin and optionally also pelargonidin. In one preferred embodiment, the composition also contains at least one trisaccharide anthocyanin. The disaccharide anthocyanins are more water-soluble than the monosaccharides; moreover, cyanidin and delphinidin anthocyanins are amongst the most water-soluble anthocyanins.

In an advantageous embodiment of the present invention anthocyanins are selected from cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

In one embodiment, the anthocyanin composition comprises delphinidin 3 glucoside (D3G).

The delphinidin-3-glucoside can be represented by the following formula:

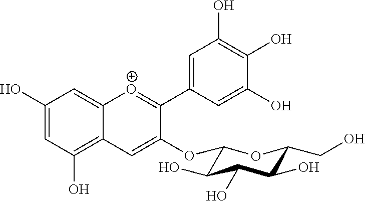

It is also intended to include pharmaceutically acceptable polymorphs, prodrugs, isomers, salts and derivatives of D3G.

The anthocyanins including the D3G can be from natural sources or from synthetic productions. Natural sources are preferably selected from fruits, flowers, leaves, stems and roots, preferably violet petal, seed coat of black soybean. Preferably anthocyanins are extracted from fruits selected from: açai, black currant, aronia, eggplant, blood orange, marion blackberry, black raspberry, raspberry, wild blueberry, cherry, queen Garnet plum, red currant, purple corn (Z. mays L.), concord grape, norton grape, muscadine grape, red cabbage, okinawan sweet potato, Ube, black rice, red onion, black carrot. Particularly suitable fruit sources for the anthocyanins are cherries, bilberries, blueberries, black currants, red currants, grapes, cranberries, strawberries, black chokeberry, and apples and vegetables such as red cabbage. Bilberries, in particular Vaccinium myrtillus, and black currants, in particular Ribes nigrum, are especially suitable. It is further preferred to use plants enriched with one or more of anthocyanins as natural sources, preferably plants enriched with delphinidin-3-rutinoside.

The counterion in the anthocyanins in the composition of the invention may be any physiologically tolerable counter anions, e.g. chloride, succinate, fumarate, malate, maleate, citrate, ascorbate, aspartate, glutamate, etc. Preferably however the counterion is a fruit acid anion, in particular citrate, as this results in the products having a particularly pleasant taste. Besides the anthocyanins, the composition may desirably contain further beneficial or inactive ingredients, such as vitamins (preferably vitamin C), flavones, isoflavones, anticoagulants (e.g. maltodextrin, silica, etc.), desiccants, etc.

It is preferred when the combined preparation, composition or antiviral agent for use comprises anthocyanins and is to be administered to the subject in a dose of the anthocyanins/regimen of 1 to 10 oral dosages of at least 80 mg anthocyanins each per day, preferably 3 to 6 oral dosages of at least 80 mg anthocyanins each per day.

Alternatively, the combined preparation, composition or antiviral agent is to be administered to the subject in 1 to 10 oral dosages of at least 20 mg D3G each per day, preferably 3 to 6 oral dosages of at least 20 mg D3G each per day.

In a further advantageous configuration, the composition is to be administered to the subject as parenteral bolus injection or infusion or parenteral nutritional solution. It is also preferred to use the composition to stabilize critical patients, where lifesaving treatments are not effective, and no last-line treatment is available (due to lack of treatment options).

The composition according to the present invention is to be administered to the subject, reaching a concentration in the target compartment at least 30 μg/ml, preferably at least 100 μg/ml. Target compartment are blood and lymph, specifically the medium surrounding the cells of the immune system, which are infected by the Herpesviridae, preferably Peripheral Blood Mononuclear Cells(PBMCs), especially B cells, T cells, dendritic cells.

It is known that viral infections can occur when a medical device is used on a subject. This is particularly the case when the device, such as a catheter or feeding tube, is to be retained in the subject for any length of time, e.g. the dwell time of the device in the subject is more than 24 hours.

Accordingly the present invention also relates to a combined preparation, composition or antiviral agent for use with a medical device which is to be inserted into the subject, or for use in a subject who has had a medical device inserted, optionally wherein the inserted device is transdermal or endotracheal. Preferably, the combined preparation, composition or antiviral agent to be administered at a site of insertion of the medical device into the subject. Alternatively, the medical device is for endotracheal intubation or parenteral nutrition. It is preferred, when the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter. It is especially preferred, when a dwell time of the medical device in the subject is more than 24 hours, more than 48 hours, more than 72 hours, more than one week, more than 2 weeks, more than 3 weeks, preferably wherein the dwell time is more than one week, more than 2 weeks or more than 3 weeks.

In a preferred embodiment, the subject is a human, preferably the subject is pregnant or immunocompromised or taking an immunosuppressant or is a carrier of a virus from the Herpesviridae family, preferably wherein the subject is a carrier of herpes simplex virus, Epstein-Barr or human cytomegalovirus.

In a preferred embodiment the subject is one who has been exposed to physical or emotional stress, or is suffering from fatigue, depression or anxiety, which may lead to reactivation of latent herpesvirus infections.

The combined preparation, composition or antiviral agent is also suitable when the subject is infected with Kaposi's sarcoma-associated herpesvirus (KSHV, HHV-8), optionally wherein the subject is HIV-positive or is suffering from AIDS.

In a preferred embodiment, the virus infection is in the liver or kidney. The tested berry extracts show a broad activity in contrast to known antivirals. Therefore, it can be for use, when a liver infection is diagnosed (EBV, CMV or HSV). Since the berry extracts shall not be toxic to kidney, it could also be used after transplantation as a prophylaxis.

Another aspect of the present invention is related to a combined preparation, composition or antiviral agent for use for the prevention or treatment of a cancer associated with a virus from the Herpesviridae family, optionally wherein:

(i) the virus is EBV and the cancer is lymphoma (including Hodgkin lymphoma and Burkitts lymphoma), nasopharyngeal cancer, gastric cancer, or breast cancer; or

(ii) the virus is HHV-8 and the cancer is Kaposi's sarcoma, primary effusion lymphoma, HHV-8-associated multicentric Castleman disease, or breast cancer.

Another aspect of the present invention is related to a combined preparation, composition or antiviral agent for the prevention or treatment of an autoimmune disease associated with a virus from the Herpesviridae family, optionally wherein:

(i) the virus is EBV and the autoimmune disease is systemic lupus erythematosus (SLE), rheumatoid arthritis (RA), Sjögren's syndrome or multiple sclerosis; or

(ii) the virus is HSV-1 and the autoimmune disease is multiple sclerosis.

In these two aspects the combined preparation, composition or antiviral agents may be as described above.

Moreover, the combined preparation, composition or antiviral agent is useful for the prevention or treatment of Alzheimer disease.

Therefore, another aspect of the invention covers a combined preparation, composition or antiviral agent for use for the prevention or treatment of Alzheimer disease, wherein the composition reduces β-amyloid plaque formation, optionally wherein the composition reduces β-amyloid plaque formation by reducing or preventing a virus infection.

The reduction of viral infection may be assessed by performing PCR on a blood sample to determine reduction in viral copy number, the viral copy number can be used to determine whether the infection is passive or active. The composition can be used both to prevent viral infection and to prevent viral reactivation.

In a specific configuration, the combined preparation, composition or antiviral agent for use for the prevention or treatment of Alzheimer disease reduces brain tissue inflammation. An encephalitis may also be prevented in this context.

In this aspect the components of the combined preparation, composition or antiviral agent may be as described above.

The invention also refers to a composition comprising an antiviral agent, and an anthocyanin composition, wherein the anthocyanin composition comprises one or more of an extract of blackcurrants an extract of bilberries an extract of red grape, and/or delphinidin 3 glucoside, preferably wherein the antiviral agent is a Herpesviridae antiviral agent, preferably wherein the antiviral agent is an inhibitor of DNA replication.

Another aspect of the present invention is related to a kit comprising in separate containers: (i) an antiviral agent; and (ii) an anthocyanin composition, wherein the anthocyanin composition comprises one or more of an extract of blackcurrants an extract of bilberries an extract of red grape, and/or delphinidin 3 glucoside, wherein the antiviral agent is a Herpesviridae antiviral agent, preferably wherein the antiviral agent is an inhibitor of DNA replication.

In particular, the components of the composition and kit can be as described above in relation to the medical use.

A further aspect of the present invention is a topical composition comprising an extract of black currants and bilberries, wherein the composition further comprises a pharmaceutically acceptable excipient suitable for a topical composition that is to be administered to the skin, preferably wherein the pharmaceutically acceptable excipient comprises one or more of a tonicity adjusting agent, a buffering agent, a preservative, an antioxidant, a stabilizer, a pH adjusting agent, a penetration enhancer, a surfactant and a humectant.

A further aspect of the present invention is an eye drop composition comprising an extract of black currants and bilberries, wherein the composition further comprises a pharmaceutically acceptable excipient suitable for a composition that is to be administered to the eye, preferably wherein the pharmaceutically acceptable excipient comprises one or more of a tonicity adjusting agent, a buffering agent, a preservative, an antioxidant, a stabilizer, a pH adjusting agent, a penetration enhancer, a surfactant and a humectant.

The present invention also refers to
a composition comprising an analgesic and an extract of black currants and bilberries, preferably wherein the analgesic is ibuprofen or paracetamol/acetaminophen,
a composition for use in treating pain associated with a virus infection in a subject, wherein the virus is from the Herpesviridae family,
a combined preparation comprising an analgesic, and an extract of black currants and bilberries, for simultaneous, separate or sequential use in medicine,
a topical composition comprising an analgesic, and an extract of black currants and bilberries,
a composition which is in the form of a topical composition or eye drops, preferably wherein the antiviral agent is acyclovir,
a combined preparation comprising an antiviral agent, and an extract of black currants and bilberries, for simultaneous, separate or sequential use in medicine.

Analgesic compounds are preferably selected from acetylsalicylic acid, Diclofenac, Dexibuprofen, Dexketoprofen, Flurbiprofen, Ibuprofen, Indometacin, Ketoprofen, Meloxicam, Nabumeton, Naproxen, Phenylbutazon, Piroxicam, Phenazon, Propyphenazon, rofecoxib, Celecoxib, Etoricoxib, Parecoxib, Metamizol, Paracetamol/Acetaminophen.

For all the compositions described above it is advantageous, when the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries. It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%. It is preferred, according to the present invention, when the extract is an alcoholic extract, preferably a methanol extract.

The present invention is also related to an agent with antiviral activity for treating or preventing a virus infection in a subject, wherein the virus is from the Herpesviridae family with a level of efficacy of 2 log levels, and an antiviral agent which is non-toxic.

The invention is also referring to an agent with antiviral activity for treating or preventing a virus infection in a subject, wherein the virus is from the Herpesviridae family with a level of efficacy of 2 log levels, which is not killing more than 30%, preferably not more than 20%, more preferably not more than 10% of cells in a cell-based assay in mammalian cells, preferably BHK cells.

This agent with antiviral activity preferably comprises one or more anthocyanins selected from cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

As noted above, the present invention is also related to a medical device suitable for insertion into a subject, the medical device comprising a coating composition on an exterior surface of the device, wherein the coating composition comprising (i) an antiviral agent; and (ii) an anthocyanin composition, wherein the anthocyanin composition comprises one or more of an extract of blackcurrants an extract of bilberries an extract of red grape, and/or delphinidin 3 glucoside, wherein the antiviral agent is a Herpesviridae antiviral agent, preferably wherein the antiviral agent is an inhibitor of DNA replication.

It is preferred, when the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter, preferably wherein the exterior surface of the medical device is plastic. It is further preferred, when a dwell time of the medical device in the subject is more than 24 hours, more than 48 hours, more than 72 hours, more than one week, more than 2 weeks, more than 3 weeks, preferably wherein the dwell time is more than one week, more than 2 weeks or more than 3 weeks.

In a preferred configuration, in the composition, kit or medical device the antiviral agent is a DNA polymerase inhibitor or a DNA terminase complex inhibitor. It is further preferred, when the antiviral agent is a nucleoside analogue or a pyrophosphate analogue, or wherein the antiviral agent is a prodrug of a nucleoside analogue or a pyrophosphate analogue. It is preferred, when the antiviral agent is acyclovir, ganciclovir, valganciclovir, foscarnet, famciclovir, penciclovir, valaciclovir, or letermovir, preferably wherein the antiviral agent is acyclovir or ganciclovir.

It is preferred, when in the composition, kit or medical device the anthocyanin composition comprises an extract of black currant and an extract of bilberries. It is further preferred, when the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries. It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%. It is preferred, according to the present invention, when the extract is an alcoholic extract, preferably a methanol extract.

In a preferred embodiment, a composition is a topical composition or eye drops, preferably wherein the composition comprises a pharmaceutically acceptable excipient suitable for a composition that is to be administered to the skin or mucous membranes or to the eye. It is further preferred, when the topical composition is a lip balm or lip protection product.

It is preferred, when the composition comprises one or more of a tonicity-adjusting agent, a buffering agent, a preservative, an antioxidant, a stabilizer, a pH adjusting agent, a penetration enhancer, a surfactant and a humectant.

The invention also covers a method of making the medical device as described, the method comprising applying the coating composition to the exterior surface of the medical device, optionally wherein the coating composition is formulated as a cream, a hydrogel cream, or a spray.

Moreover, the invention refers to a deep-lung particle comprising a composition comprising an anthocyanin composition and an antiviral agent, wherein the anthocyanin composition comprises one or more of an extract of black currants, an extract of bilberries, an extract of red grapes, and/or delphinidin 3 glucoside, which is dispensed into the deeper respiratory tract of an individual and a device for dispensing a deep-lung particle into the deeper respiratory tract of an individual.

The composition may comprise a formulation of an extract of black currants, an extract of bilberries, an extract of red grapes, and/or delphinidin 3 glucoside with nanoparticles, preferably liposomes. Such formulations may be inhaled to maximize the delivery of nanoparticles into the lung. Inhalation facilitates the localized delivery of compositions directly to the lungs via the oral or nasal inhalation route. For example, aerosolized delivery of liposomal interleukin-2 (IL-2) in dogs has been shown to be effective against pulmonary metastases from osteosarcoma (Khanna C, Anderson P M, Hasz D E, Katsanis E, Neville M, Klausner J S. Interleukin-2 liposome inhalation therapy is safe and effective for dogs with spontaneous pulmonary metastases. Cancer 1997; 79: 1409-21.) Moreover, the delivery of anticancer drugs via nanoparticles has been shown to be efficacious and safe in a variety of cancers. Anticancer drugs can also be formulated into drug nanocrystals with high drug loading and minimal use of excipients. (Sharad M, Wei G, Tonglei L, Qi Z, Review: Pulmonary delivery of nanoparticle chemotherapy for the treatment of lung cancers: challenges and opportunities, Acta Pharmacologica Sinica (2017) 38: 782-797).

In a preferred embodiment, a nanoparticle suspension comprising the composition according to the present invention is aerosolized into droplets with appropriate aerodynamic diameters using currently available inhalation devices. Such inhalation devices are preferably selected from nebulizers and pressurized metered dose inhalers (pMDI).

Therefore, in an advantageous configuration, the composition according to the present invention may also be formulated as nanoparticle suspension for use in a nebulizer. Such nebulizers convert suspension of nanoparticles into inhalable droplets and may be used for the delivery of the composition into the deep lungs without compromising liposome integrity. An alternative configuration refers to pMDIs, which create small inhalable droplets of drugs suspended in compressed propellant (such as hydrofluoroalkane (HFA)).

The present invention also refers to a nanoparticle formulation as a dry powder, which offers greater long-term stability than a suspension. Controlling the size of nanoparticles is central for their formulation into reliable and efficient inhalable dry powders. Nanoparticles can be dried with/without excipients via spray-drying, freeze-drying and spray freeze-drying to generate stable and uniformly sized inhalable particles.

In an alternative embodiment, nanoparticles may be co-dried with excipients, which leads to the formation of inhalable nanoparticle aggregates in an excipient matrix. It is possible to utilize particle engineering and ensure consistent and highly efficient delivery of nanoparticles to the lungs through nano-aggregates, large porous particles, and other formulation techniques.

The activity of the combination of the invention may also be utilized in the context of cell culture and cell storage ex vivo, and in particular in the preparation of cells for cell therapy. Accordingly, the present invention also provides a method for preventing or reducing the risk of a virus infection in a cell or cells ex vivo comprising contacting the cell or cells with a composition comprising: (i) an antiviral agent; and (ii) an anthocyanin composition, wherein the anthocyanin composition comprises one or more of an extract of blackcurrants an extract of bilberries an extract of red grape, and/or delphinidin 3 glucoside, and wherein the antiviral agent is a Herpesviridae antiviral agent, preferably wherein the antiviral agent is an inhibitor of DNA replication.

Optionally the cell or cells are stem cells or CAR T cells, optionally wherein the contacting comprises culturing or storing the cell or cells with the composition.

Item List

Preferred embodiments of the present invention are summarized in the following item list:
1. A combined preparation comprising an anthocyanin composition and an antiviral agent for use in treating or preventing a virus infection in a subject, wherein the anthocyanin composition comprises one or more of an extract of black currants, an extract of bilberries, an extract of red grapes, and/or delphinidin 3 glucoside, wherein the virus is from the Herpesviridae family and the antiviral agent is a Herpesviridae antiviral agent, and wherein the anthocyanin composition and the antiviral agent are for simultaneous, separate or sequential use.
2. The combined preparation for use according to item 1, wherein the anthocyanin composition comprises an extract of blackcurrants and bilberries.
3. The combined preparation for use according to item 1 or item 2, wherein the combined preparation is a composition comprising the anthocyanin composition and the antiviral agent.
4. An anthocyanin composition comprising one or more of an extract of black currants, an extract of bilberries, an extract of red grapes, and/or delphinidin 3 glucoside for use in treating or preventing a virus infection in a subject, wherein the virus is from the Herpesviridae family and the antiviral agent is a Herpesviridae antiviral agent, and wherein the composition is to be administered in combination with an antiviral agent active against the virus, or wherein the composition is to be administered to a subject treated with the antiviral agent.
5. An anti-viral agent for use in treating or preventing a virus infection in a subject, wherein the virus is from the Herpesviridae family and the antiviral agent is a Herpesviridae antiviral agent, and wherein the antiviral agent is to be administered: (i) in combination with an anthocyanin composition comprising one or more of an extract of blackcurrants, an extract of bilberries, an extract of red grapes, or delphinidin 3 glucoside active against the virus; or (ii) to a subject treated with the anthocyanin composition.
6. The combined preparation, composition or antiviral agent for use according to any preceding item, wherein the antiviral agent is an inhibitor of DNA replication, optionally wherein the antiviral agent is a DNA polymerase inhibitor or a DNA terminase complex inhibitor.
7. The combined preparation, composition or antiviral agent for use according to any preceding item, wherein the antiviral agent is a nucleoside analogue or a pyrophosphate analogue, or wherein the antiviral agent is a prodrug of a nucleoside analogue or a pyrophosphate analogue.
8. The combined preparation, composition or antiviral agent for use according to any preceding item, wherein the antiviral agent is one or more of acyclovir, ganciclovir, valganciclovir, foscarnet, famciclovir, penciclovir, or valaciclovir, or letermovir, preferably wherein the antiviral agent is acyclovir.
9. The combined preparation, composition or antiviral agent for use ac according to any preceding item, wherein the black currants are the fruit of Ribes nigrum and/or the bilberries are the fruit of Vaccinium myrtillus and preferably, wherein the composition contains an extract from black currants and bilberries in a weight ratio of 0.5:1 to 1:0.5.
10. The combined preparation, composition or antiviral agent for use according to any preceding item, wherein the extract is an extract of the pomaces from blackcurrants and/or bilberries and/or red grapes.
11. The composition for use according to any preceding item, wherein the composition comprises anthocyanins and the anthocyanins are present in the composition at a concentration of at least 25 weight-%.
12. The combined preparation, composition or antiviral agent for use according to any preceding item, wherein the extract is an alcoholic extract, preferably a methanol extract.
13. The composition for use according to any preceding item, wherein the extract is prepared by a process comprising the steps of extraction of black currants and/or bilberries, purification via chromatography, mixing of the extract(s) with water and spray-drying of the mixture.
14. The combined preparation, composition or antiviral agent for use according to any preceding item, wherein the composition comprises one or more of the following anthocyanins:
   cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside,
   preferably comprises cyanidin-3-glucoside, cyanidin-3-rutinoside, delphinidin-3-glucoside, delphinidin-3-rutinoside, cyanidin-3-galactoside and delphinidin-3-galactoside, more preferably wherein the extract comprises delphinidin-3-glucoside.
15. The combined preparation, composition or antiviral agent for use according to any preceding item, wherein the virus is herpes simplex virus-1 (HSV-1), herpes simplex virus-2 (HSV-2), Varicella zoster virus (VZV), Epstein-Barr virus (EBV), Cytomegalovirus (CMV), Roseolovirus, or Kaposi's sarcoma-associated herpesvirus (KSHV, HHV-8), preferably HSV-1, EBV, CMV, and HHV-8, more preferably HSV-1.
16. The combined preparation, composition or antiviral agent for use according to any preceding item, wherein: (i) the virus is CMV and the antiviral agent is ganciclovir; or (ii) the virus is HSV-1 or EBV and the antiviral agent is acyclovir.
17. The composition for use according to any preceding item, wherein the composition comprises anthocyanins and is to be administered to the subject in 1 to 10 oral dosages of at least 80 mg anthocyanins each per day, preferably 3 to 6 oral dosages of at least 80 mg anthocyanins each per day.
18. The combined preparation, composition or antiviral agent for use according to any preceding item, wherein the composition is to be administered to the subject in 1 to 10 oral dosages of at least 20 mg D3G each per day, preferably 3 to 6 oral dosages of at least 20 mg D3G each per day.
19. The combined preparation, composition or antiviral agent for use according to any preceding item, wherein the extract is to be administered to the subject, reaching a concentration in the target compartment of at least 30 μg/ml, preferably at least 100 μg/ml.
20. The combined preparation, composition or antiviral agent for use according to any preceding item, wherein the composition is for use with a medical device which is to be inserted into the subject, or wherein the subject has had a medical device inserted, optionally wherein the inserted device is transdermal or endotracheal.
21. The combined preparation, composition or antiviral agent for use according to item 20, wherein the composition is to be administered at a site of insertion of the medical device into the subject.
22. The combined preparation, composition or antiviral agent for use according to item 20 or 21, wherein the medical device is for endotracheal intubation or parenteral nutrition.
23. The combined preparation, composition or antiviral agent for use according to any of item 20 to 22, wherein the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter.
24. The combined preparation, composition or antiviral agent for use according to any of item 20 to 23, wherein a dwell time of the medical device in the subject is more than 24 hours, more than 48 hours, more than 72 hours, more than one week, more than 2 weeks, more than 3 weeks, preferably wherein the dwell time is more than one week, more than 2 weeks or more than 3 weeks.
25. The combined preparation, composition or antiviral agent for use according to any preceding item wherein the subject is a human, preferably wherein the subject is pregnant or immunocompromised or the subject is taking an immunosuppressant.
26. The combined preparation, composition or antiviral agent for use according to any preceding item, wherein the subject is a carrier of a virus from the Herpesviridae family, preferably wherein the subject is a carrier of herpes simplex virus.
27. The combined preparation, composition or antiviral agent for use according to any preceding item, wherein the subject is infected with Kaposi's sarcoma-associated herpesvirus (KSHV, HHV-8), optionally wherein the subject is HIV-positive or is suffering from AIDS.
28. The combined preparation, composition or antiviral agent for use according to any preceding item, wherein the virus infection is in the liver or kidney.
29. The combined preparation, composition or antiviral agent for use according to any preceding item for the prevention or treatment of a cancer associated with a virus from the Herpesviridae family, optionally wherein:
   (i) the virus is EBV and the cancer is lymphoma (including Hodgkin lymphoma and Burkitts lymphoma), nasopharyngeal cancer, gastric cancer, or breast cancer; or
   (ii) the virus is HHV-8 and the cancer is Kaposi's sarcoma, primary effusion lymphoma, HHV-8-associated multicentric Castleman disease, or breast cancer.
30. The combined preparation, composition or antiviral agent for use according to any preceding item for the prevention or treatment of an autoimmune disease associated with a virus from the Herpesviridae family, optionally wherein:
   (i) the virus is EBV and the autoimmune disease is systemic lupus erythematosus (SLE), rheumatoid arthritis (RA), Sjögren's syndrome or multiple sclerosis; or
   (ii) the virus is HSV-1 and the autoimmune disease is multiple sclerosis.
31. The combined preparation, composition or antiviral agent for use according to the preceding item for the prevention or treatment of Alzheimer disease.
32. The combined preparation, composition or antiviral agent for use according to item 31, which reduces β-amyloid plaque formation, optionally which reduced β-amyloid plaque formation by reducing or preventing an active virus infection.
33. The combined preparation, composition or antiviral agent for use according to item 31 or claim 32, which reduces brain tissue inflammation.
34. A composition comprising an antiviral agent, and an anthocyanin composition, wherein the anthocyanin composition comprises one or more of an extract of blackcurrants an extract of bilberries an extract of red grape, and/or delphinidin 3 glucoside, wherein the antiviral agent is a Herpesviridae antiviral agent, preferably wherein the antiviral agent is an inhibitor of DNA replication.
35. A kit comprising in separate containers: (i) an antiviral agent; and (ii) an anthocyanin composition, wherein the anthocyanin composition comprises one or more of an extract of blackcurrants an extract of bilberries an extract of red grape, and/or delphinidin 3 glucoside, wherein the antiviral agent is a Herpesviridae antiviral agent, preferably wherein the antiviral agent is an inhibitor of DNA replication.
36. A medical device suitable for insertion into a subject, the medical device comprising a coating composition on an exterior surface of the device, wherein the coating composition comprising (i) an antiviral agent; and (ii) an anthocyanin composition, wherein the anthocyanin composition comprises one or more of an extract of blackcurrants an extract of bilberries an extract of red grape, and/or delphinidin 3 glucoside, wherein the antiviral agent is a Herpesviridae antiviral agent, preferably wherein the antiviral agent is an inhibitor of DNA replication.
37. The medical device according to item 36, wherein the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter, preferably wherein the exterior surface of the medical device is plastic.
38. The composition, kit or medical device of any of items 34 to 37, wherein the antiviral agent is a DNA polymerase inhibitor or a DNA terminase complex inhibitor.
39. The composition, kit or medical device of any of items 34 to 38, wherein the antiviral agent is a nucleoside analogue or a pyrophosphate analogue, or wherein the antiviral agent is a prodrug of a nucleoside analogue or a pyrophosphate analogue.
40. The composition, kit or medical device of any of items 34 to 39, wherein the antiviral agent is acyclovir, ganciclovir, valganciclovir, foscarnet, famciclovir, penciclovir, valaciclovir, or letermovir, preferably wherein the antiviral agent is acyclovir or ganciclovir.
41. The composition, kit or medical device according to any of items 34 to 40, wherein the anthocyanin composition comprises an extract of black currant and an extract of bilberries.
42. The composition according to any of items 34, 38 to 41 which is a topical composition or eye drops, preferably wherein the composition comprises a pharmaceutically acceptable excipient suitable for a composition that is to be administered to the skin or mucous membranes or to the eye.
43. The composition, kit or medical device according to any of items 34 to 42, wherein the antiviral agent is acyclovir.
44. The composition, kit or medical device according to any of items 34 to 42, wherein the antiviral agent is ganciclovir.
45. The composition according to any of items 34 or 38 to 44, which comprises one or more of a tonicity-adjusting agent, a buffering agent, a preservative, an antioxidant, a stabilizer, a pH adjusting agent, a penetration enhancer, a surfactant and a humectant.
46. The composition according to any of items 34 or 38 to 45, which comprises an analgesic.
47. The composition, kit or medical device according to any of items 34 to 46, wherein the anthocyanins are present in the composition at a concentration of at least 25 weight-%.
48. The composition, kit or medical device according to any of items 34 to 47, comprising at least 50 wt % extract.
49. A method for treating or preventing a virus infection, or preventing virus reactivation, in a subject in need thereof comprising administering to the subject an effective amount of an anthocyanin composition and an antiviral agent, wherein the anthocyanin composition comprises one or more of an extract of black currants, an extract of bilberries, an extract of red grapes, and/or delphinidin 3 glucoside, wherein the virus is from the Herpesviridae family, wherein the antiviral agent is a Herpesviridae antiviral agent.
50. A method for preventing a device-associated virus infection in a subject, comprising: (a) inserting a device into the subject and administering an effective amount of an anthocyanin composition and an antiviral agent; and/or (b) applying an effective amount of an anthocyanin composition and an antiviral agent to an external surface of a device and inserting the device into the subject, wherein the anthocyanin composition and the antiviral agent are as defined in item 49, and wherein the virus is from the Herpesviridae family.
51. A method for treating or preventing a cancer associated with a virus from the Herpesviridae family in a subject in need thereof, comprising administering to the subject effective amounts of an anthocyanin composition and an antiviral agent, wherein the anthocyanin composition and the antiviral agent are as defined in item 49.
52. A method for treating or preventing an autoimmune disease associated with a virus from the Herpesviridae family in a subject in need thereof, comprising administering to the subject effective amounts of an anthocyanin composition and an antiviral agent, wherein the anthocyanin composition and the antiviral agent are as defined in item 49.
53. A method for reducing β-amyloid plaque formation and/or reducing brain tissue inflammation in a subject in need thereof, comprising administering to the subject an effective amount of an anthocyanin composition and an antiviral agent, wherein the anthocyanin composition and the antiviral agent are as defined in item 49, optionally wherein the combination reduces β-amyloid plaque formation and/or brain tissue inflammation by reducing or preventing an infection by a virus from the Herpesviridae family.
54. The method according to any of items 49 to 53 wherein the anthocyanin composition is as defined in any of items 9 to 14 and/or the antiviral agent is as defined in any of items 6 to 8.
55. The method according to any of items 49 to 54, wherein the virus is as defined in item 15.
56. The method according to any of items 49 to 55, wherein the composition is to be administered as defined in any of items 17 to 19.
57. The method according to any one of items 49 to 56, wherein the subject is as defined in any of items 25 to 27.

### EXAMPLES

The berry extracts composition (Healthberry® 865; Evonik Nutrition & Care GmbH, Darmstadt, Germany) used in the present study is a dietary supplement consisting of 17 purified anthocyanins (all glycosides of cyanidin, peonidin, delphinidin, petunidin, and malvidin) isolated from black currant (Ribes nigrum) and bilberries (Vaccinium myrtillus).

The relative content of each anthocyanin in the Healthberry® 865 product was as follows: 33.0% of 3-O-b-rutinoside, 3-O-b-glucosides, 3-O-b-galactosides, and 3-O-b-arabinosides of cyanidin; 58.0% of 3-O-b-rutinoside, 3-O-b-glucosides, 3-O-b-galactosides, and 3-O-b-arabinosides of delphinidin; 2.5% of 3-O-b-glucosides, 3-O-b-galactosides, and 3-O-b-arabinosides of petunidin; 2.5% of 3-O-b-glucosides, 3-O-b-galactosides, and 3-O-b-arabinosides of peonidin; 3.0% of 3-O-b-glucosides, 3-0-b-galactosides, and 3-O-b-arabinosides of malvidin.

The 3-O-b-glucosides of cyanidin and delphinidin constituted at least 40-50% of the total anthocyanins.

The major anthocyanins contained in the berry extract used are cyanidin-3-glucoside, cyanidin-3-rutinoside, delphinidin-3-glucoside, delphinidin-3-rutinoside, cyanidin-3-galactoside and delphinidin-3-galactoside.

In addition to the anthocyanins mentioned above, the product also contained maltodextrin (around 40 weight-% of the composition), and citric acid (to maintain stability of anthocyanins). The amount of anthocyanin citrate is at least 25 weight-% of the composition. The composition is prepared from black currants and bilberries by a process comprising the steps of alcoholic extraction of black currants and bilberries, purification via chromatography, mixing of the extracts with maltodextrin citrate and water and spray-drying of the mixture. The product composition contains extracts of black currants and bilberries mixed in a weight ratio of around 1:1.

Materials:

TABLE 1
Materials used for the measurement of cell survival and metabolism

| Material | Supplier |
|---|---|
| RealTime-Glo ™ MT Cell | Promega GmbH, Mannheim (Germany) |
| Viability Assay | |
| CellTiter-Glo ™ | Promega GmbH, Mannheim (Germany) |
| Luminescent | |
| Cell Viability Assay | |
| Dulbecco's Modified | Gibco Life technologies, Carlsbad (USA) |
| Eagle's medium (DMEM) | |
| Fetal bovine serum | Gibco Life technologies, Carlsbad (USA) |
| BHK cells | ATCC/American Type Culture Collection |
| | in Partnership with LGC standards, Wesel |
| | (Germany) |
| Healthberry ® 865 | Evonik Nutrition & Care GmbH, |
| (anthocyanin content | Darmstadt (Germany) |
| 29.7%) | |

TABLE 2
Devices used for the measurement of cell survival and metabolism.

| Device | Supplier |
|---|---|
| Centro LB 960 microplate | Berthold Technologies, (Germany) |
| luminometer | |

TABLE 3
Materials used for anti-viral assay

| Material | Supplier |
|---|---|
| Wildtype HSV-1 virus, herpes simplex | Institute of Virology, Wiirzburg (Germany) |
| virus 1 | |
| Influenza virus serotype A | patient derived isolate, Institute of Virology |
| | Wurzburg (Germany) |
| Dulbecco's Modified Eagle's medium | Gibco Life technologies, Carlsbad (USA) |
| (DMEM) | |
| Fetal bovine serum | Gibco Life technologies, Carlsbad (USA) |
| BHK cells | ATCC/American Type Culture Collection in |
| | Partnership with LGC standards, Wesel (Germany) |
| MDCK cells | ATCC/American Type Culture Collection in |
| | Partnership with LGC standards, Wesel (Germany) |
| HP Viral Nucleic Acid Kit | Hoffman-La-Roche Ltd., Basel (Switzerland) |
| RTqPCR LightMix ® Modular Influenza | Hoffman-La-Roche Ltd., Basel (Switzerland) |
| A kit (Cat. No. 07 792 182 001) | |
| LightCycler ® Multiplex RNA Virus | Hoffman-La-Roche Ltd., Basel (Switzerland) |
| Master kit (Cat. No. 07 083 173 001) | |
| Healthberry ® 865 (anthocyanin | Evonik Nutrition & Care GmbH, Darmstadt |
| content 29.7%) | (Germany) |
| Bilberry extract, Vaccinium myrtillus | Evonik Nutrition & Care GmbH, Darmstadt |
| (anthocyanin content 38.8%) | (Germany) |
| Black currant extract, Ribes nigrum | Evonik Nutrition & Care GmbH, Darmstadt |
| (anthocyanin content 30%) | (Germany) |
| Berry extract analogue to | Evonik Nutrition & Care GmbH, Darmstadt |
| Healthberry ® 865 without maltodextrin | (Germany) |
| GLUCIDEX IT 19 (maltodextrin) | ROQUETTE GmbH, Frankfurt (Germany) |
| Delphinidin 3-rutinoside/D3R | Polyphenols AS, Sandnes (Norway) |
| Delphinidin 3-glucoside/D3G | Polyphenols AS, Sandnes (Norway) |
| Cyanidin 3-rutinoside/C3R | Polyphenols AS, Sandnes (Norway) |
| Cyanidin 3-glucoside/C3G | Polyphenols AS, Sandnes (Norway) |
| Delphinidin 3-galactoside/D3Gal | Polyphenols AS, Sandnes (Norway) |
| Petunidin 3-glucoside/Pet3G | Polyphenols AS, Sandnes (Norway) |

TABLE 4
Devices used for the anti-viral assay

| Device | Supplier |
|---|---|
| LightCycler96 qPCR 20 machine | Hoffman-La-Roche Ltd., |
| | Basel (Switzerland) |
| Lighcylcler96 Application software | Hoffman-La-Roche Ltd., |
| V1.1 | Basel (Switzerland) |
| Perkin Elmer Ensight system | Perkin Elmer, Rodgau (Germany) |

Methods:

Test Compound Preparation:

All test compounds were dissolved and diluted in cell culture medium. The overall amount of anthocyanins was normalized between Healthberry® 865 and the single anthocyanins (e.g. 500 μg/mL of Healthberry® 865 corresponds to 150 μg/mL of anthocyanins tested for the single test compounds) or as well the single berry extracts (taken into account that Healthberry® 865 also contains maltodextrin besides the anthocyanins). The medium served as control for viral inhibition or cytotoxicity.

Cell Viability Assay:

Cell viability was measured by RealTime-Glo™ MT Cell Viability Assay (Cat. No. G9712, Promega, Germany). BHK cells were incubated with decreasing amounts of the compound solubilized in DMEM. Wells with DMEM alone served as control. The MT Cell Viability Substrate and the NanoLuc® luciferase were added according to the manufacturer's instructions. The assays were performed in triplicates. After 3 days the luminescence signal was measured with Centro LB 960 microplate luminometer (Berthold Technologies, Germany). Luminescence values after 1 h were set to 1 and changes over time were determined.

Anti-Viral Assay:

Herpes Virus Infection:

BHK cells were incubated with decreasing concentration of the solubilized test compounds for approx. 1 h. All concentrations were analyzed by six independent replicates on a black 96 well plate (PerkinElmer). Cells were infected with GFP-encoding wildtype HSV-1 virus and incubated for two days. Two days after infection, HSV-1-infected cells and GFP expressing cells were directly counted using the PerkinElmer Ensight system with optical cell culture plates. The instrument was controlled by manual counting.

To not only analyze the virus entry and early phase of virus replication of infection but also later phases of viral replication, the test assay was adjusted accordingly. BHK cells were incubated with test compounds and subsequently infected with HSV-1. Two days after infection supernatants were collected, centrifuged to remove detached cells and used to infect BHK cells. After two additional days infected cells were quantified using the Ensight system.

Furthermore, as preliminary test Aciclovir concentrations (0.5-2 μg/mL) were titrated corresponding to each virus preparation before the combination treatment assays.

From the first identification till now, antiviral compounds are initially identified via screening assay either in vitro or in cell culture using replication assays. Even the activities of compounds identified by in vitro enzyme screening tests need to be verified in cell culture-based assays. These assays are state of the art methods to identify and confirm antiviral activities since they allow the quantification of the inhibition of viral replication and ensure the cellular uptake of compounds. For example, aciclovir, the gold standard in the treatment of HSV-1, was identified by screening of antiviral substances in sponges (Elion et al., 1977 Selectivity of action of an antiherpetic agent, 9-(2-hydroxyethoxymethyl)guanine. PNAS 74. 5716). Later, the antiviral activity of aciclovir inhibiting other members of the Herpesviridae was shown in cell culture-based assays as well (AKESSON-JOHANSSON et al., 1990 Inhibition of Human Herpesvirus 6 Replication b y9-[4-Hydroxy-2-(Hydroxymethyl)Butyl]Guanine (2HM-HBG) and Other Antiviral Compounds. AAC 34. 2417). Moreover, all compounds used as clinical drugs against HIV-1, such as 3TC and Lopinavir (ABT-378), were initially tested in vitro to demonstrate their antiviral effects (Coates et al., 1992. The Separated Enantiomers of 2′-Deoxy-3′-Thiacytidine (BCH 189) Both Inhibit Human Immunodeficiency Virus Replication In Vitro. AAC 36. 202; Sham et al. 1998. ABT-378, a Highly Potent Inhibitor of the Human Immunodeficiency Virus Protease. AAC 42. 3218).

Influenza Genome Determination:

MDCK cells were seeded in 48 well plates. After 24 h test compounds were added, and cells were subsequently infected with influenza A virus. All infections were performed in triplicates. Cell culture supernatants were harvested three days post-infection and centrifuged at 2000 rpm to remove detached cells and analyze viruses secreted to the supernatant. Viral RNAs were isolated from 200 μL cell culture supernatants using the Roche HP Viral Nucleic Acid Kit according to the manufacturer's manual. Viral genome copy numbers were determined using 5 μL of the eluted RNA and the RTqPCR LightMix® Modular Influenza A kit (Cat. No. 07 792 182 001, Roche) in combination with the LightCycler® Multiplex RNA Virus Master kit (Cat. No. 07 083 173 001, Roche). All PCR reactions were performed in triplicates from a RNAs with a Roche LightCycler96 qPCR 20. The Cq values were determined with the respective cycler software (Roche Lighcylcler96 Application software V1.1). The internal standard of the Modular Influenza A kit with 1000 genome copies served as positive control. Quality was ensured by following the MIQE guidelines.

### Example 1: Influence of Berry Extracts on Cell Viability

To exclude cellular toxicity and adverse side effects, cellular viabilities of the test compounds on BHK cells (96-well-plate: 650 cells/well) were determined with the RealTime-Glo™ MT Cell Viability Assay kit. This assay measures the intracellular ATP content and therefore provides information on the cellular viability and metabolism. The cells were incubated with decreasing compound concentration in triplicate assays. Subsequently, both the MT Cell Viability Substrate and NanoLuc® Enzyme were added, and the luciferase activities were measured after 1 h. The luminescence was measured after three days and normalized on the mean of the medium control wells. These compensations result in values of 1 for the medium control and values less than 1 indicate a lower number of cells or a decrease in metabolic activity compared to the appropriate controls.

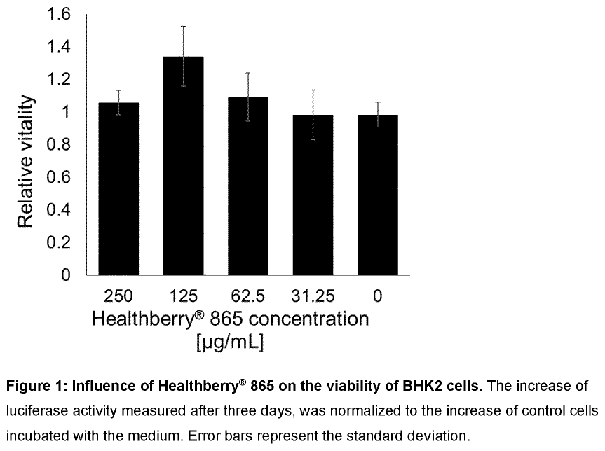

FIG. 1 displays the influence of Healthberry® 865 on the viability of BHK2 cells. The increase of luciferase activity measured after three days, was normalized to the increase of control cells incubated with the medium. Error bars represent the standard deviation.

Healthberry® 865 did not negatively influence cellular growth or metabolic activity at any concentration analysed, indicating the compound was non-toxic at these concentrations.

### Example 2: Anti-Viral Effects of Healthberry® 865 on Herpes Simplex Virus 1

BHK cells were pre-incubated with decreasing concentrations of either Healthberry® 865 or with Healthberry® 865 without maltodextrin. The concentrations of material without maltodextrin were adjusted to 0.6 times of the sugar containing product to compensate for the 40% maltodextrin content of Healthberry® 865. Thus, comparable concentrations of anthocyanins were used. The cells were subsequently infected with GFP-encoding HSV at a multiplicity of infection of 2.5, and infected GFP-expressing cells were counted one day after infection using the PerkinElmer Ensight system. Both Healthberry® 865 and the berry extract analogue without maltodextrin suppressed viral infectivity about 2 log steps at Healthberry® 865 concentrations of >0.250 μg/mL. This inhibition of viral suppression observed is in the range of common anti-viral pharmaceutical compounds and indicates that Herpes simplex is a prime target for berry extracts of black currants and bilberries, such as Healthberry® 865. The analysis of berry extract analogue without maltodextrin showed that a concentration of 150 μg/mL of the active substances (corresponding to 250 μg/mL Healthberry® 865) is sufficient for the suppression of HSV. Thus, the sugar is not required as potential co-factor for drug uptake.

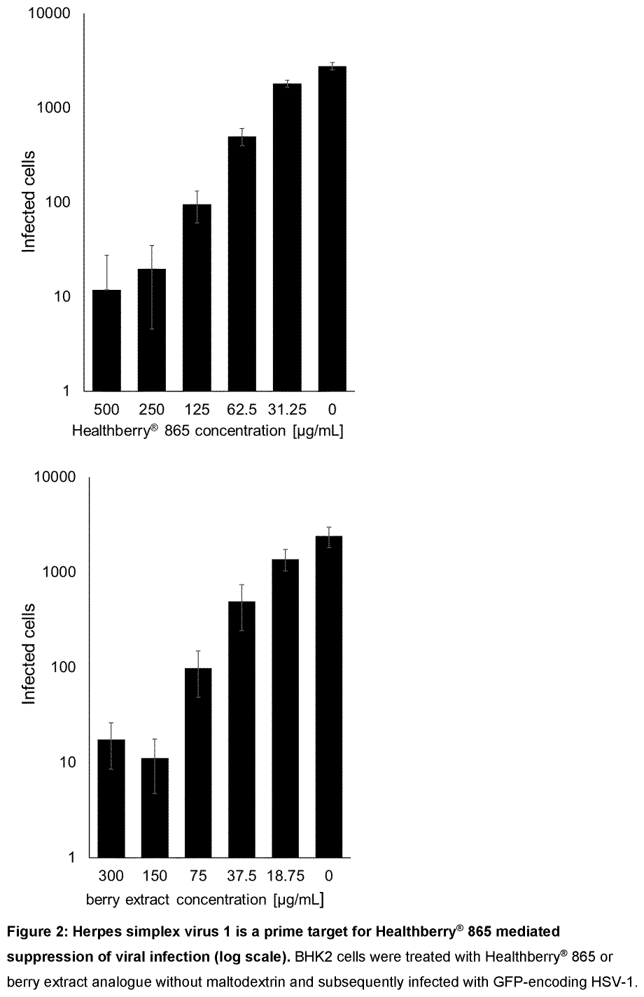

FIG. 2 shows that Herpes simplex virus 1 is a prime target for Healthberry® 865 mediated suppression of viral infection (log scale). BHK2 cells were treated with Healthberry® 865 or berry extract analogue without maltodextrin and subsequently infected with GFP-encoding HSV-1.

### Example 3: Anti-Viral Effects of Berry Extracts on Influenza a Virus (Comparative)

The influence of Healthberry® 865 and single anthocyanins on the replication of Influenza A virus were analyzed. MDCK cells were incubated with the test compounds and subsequently infected with a patient-derived isolate of Influenza virus serotype A. All reactions were performed in triplicates. Cell culture supernatants were harvested after three days, and viral genomic RNAs were isolated from 200 μL cell culture supernatants. Viral loads were determined by RTqPCR using the LightMix® Modular Influenza A kit (Roche). Positive controls with 1000 Influenza genome copies were included in the RTqPCR. All RTqPCR reactions were performed in triplicates.

All test materials, including Healthberry® 865, showed similar amounts of virus in the supernatant as the negative control, with only minor differences indicating that none of the components inhibited influenza virus replication.

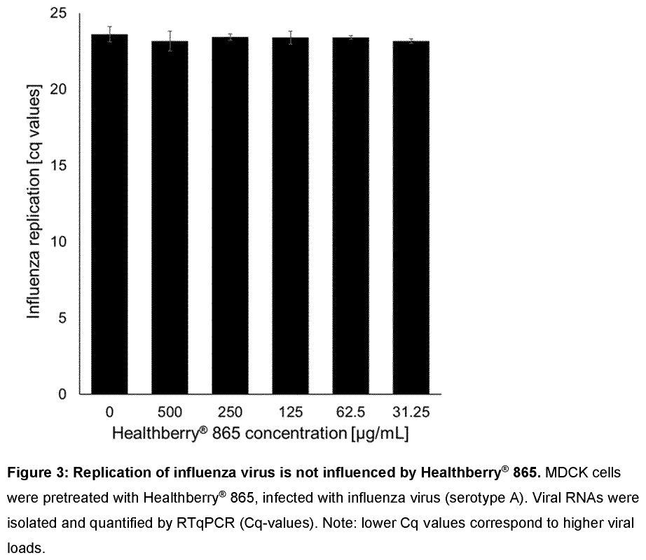

FIG. 3 shows that the replication of influenza virus is not influenced by Healthberry® 865. MDCK cells were pretreated with Healthberry® 865, infected with influenza virus (serotype A). Viral RNAs were isolated and quantified by RTqPCR (Cq-values; note: lower Cq values correspond to higher viral loads).

The results displayed no effect of Healthberry® 865 on Influenza A virus confirming the specificity of the anti-viral effects of berry extracts of black currants and bilberries on specific viruses or virus families, respectively. Other compounds as the single anthocyanins also did not show any influence on the replication of influenza virus.

### Example 4: Anti-Viral Effects of Berry Extracts on Herpes Simplex Virus 1

Since Healthberry® 865 is a composition of bilberry and black currant extracts, it was analyzed, whether both extracts contain the compound active against HSV-1. BHK cells were incubated with 500, 250, and 125 mg/mL of Healthberry® 865, bilberry or black currant extract followed by infection with HSV-1. Two days after infection supernatants were collected, centrifuged to remove detached cells and used to infect BHK cells. After two additional days infected cells were quantified using the PerkinElmer Ensight system. The mean of infected cells from six independent wells was calculated. Error bars show the standard deviation.

Besides Healthberry® 865 both extracts showed viral inhibition indicating that the active compounds are present in both bilberry and black currant extracts. But in direct comparison with Healthberry® 865, bilberry and black currant extracts suppressed the HSV-1 viral infection to a lesser extent than Healthberry® 865, although especially the bilberry extract even contains about 10% more anthocyanins than Healthberry® 865. Especially in higher concentrations like 500 μg/mL bilberry and black currant extracts reached about 1.5 log scale reduction of viral infection whereas Healthberry® 865 surprisingly reached up to 2-3 log scales. The absolute values of infected cells emphasized the significance of the effect even more, with Healthberry® 865 reducing the number of infected cells from about 1 million to ˜300 (decrease to ˜0.3%), whereas the single extracts only reduce about 90000 infected cells down to 2200-3500 (decrease to ˜3%).

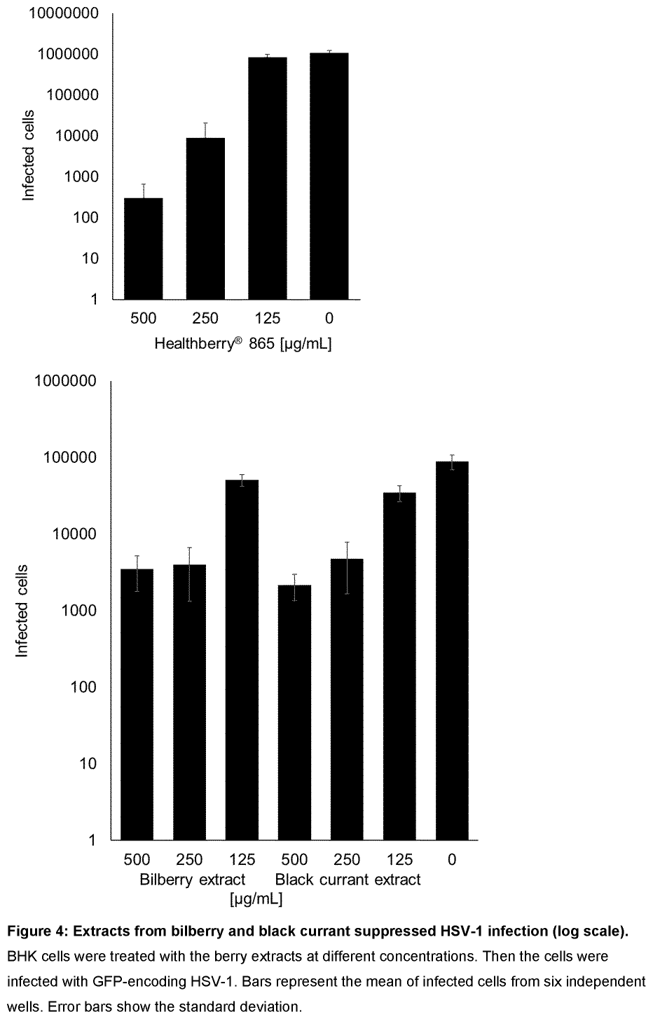

FIG. 4 shows that berry extracts from bilberry and black currant mediated suppression of viral infection (log scale). BHK cells were treated with black currant or bilberry extract and subsequently infected with GFP-encoding HSV-1.

### Example 5: Anti-Viral Effects of Anthocyanins on Herpes Simplex Virus 1

To further identify the active compound of Healthberry® 865 several known anthocyanins were tested. Neither C3G nor D3Gal or Pet3G inhibited HSV-1, while D3G decreased viral infectivity like Healthberry® 865 providing evidence that D3G is an active HSV-1 inhibitor.

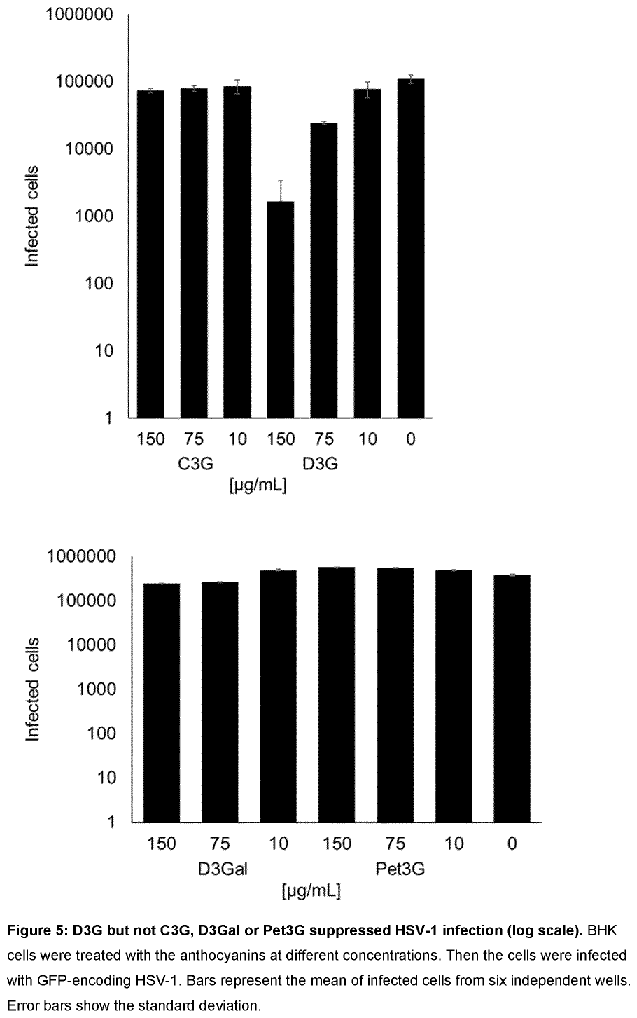

FIG. 5 shows that D3G, but not C3G, D3Gal or Pet3G, mediated suppression of viral infection (log scale). BHK cells were treated with anthocyanins and subsequently infected with GFP-encoding HSV-1.

### Example 6: Synergistic Effect of Healthberry® 865 and Aciclovir on Herpes Simplex Virus 1

Previous results have shown that Healthberry® 865 acts on the HSV-1 replication between virus entry and the early phase of virus replication since GFP-expression as readout is active in the early phase of viral gene expression. However, Aciclovir, the standard therapy for HSV-1, targets viral DNA replication before the late phase of virus replication cycle. Based on these aspects it was analyzed whether Aciclovir and Healthberry® 865 synergistically inhibit virus replication. BHK cells were incubated either with Healthberry® 865 alone or with Healthberry® 865 in combination with 0.5 μg/ml Aciclovir and subsequently infected with HSV-1. Two days after infection supernatants were collected, centrifuged to remove detached cells and used to infect BHK cells. After two addition days infected cells were quantified.

The results show, that the treatment with a standard test concentration of 0.5 μg/ml Aciclovir resulted in a reduction of infected cells of two orders of magnitude, whereas Healthberry® 865 alone in the concentration of 500 μg/mL reduced viral infectivity by more than three orders of magnitude. Furthermore, a combination of Aciclovir and Healthberry® 865 showed synergistic effects represented especially by lower doses of Healthberry® 865, like 125 μg/mL and 250 μg/mL, which achieved the reduction of infected cells as well of three orders of magnitude when combined with Aciclovir (absolute values display a reduction from 1 million infected cells to less than thousand). These experiments provided evidence that Healthberry® 865 is more effective on a complete replication cycle and that Aciclovir acts synergistically with Healthberry® 865 opening the opportunity that patients could benefit from a combination therapy.

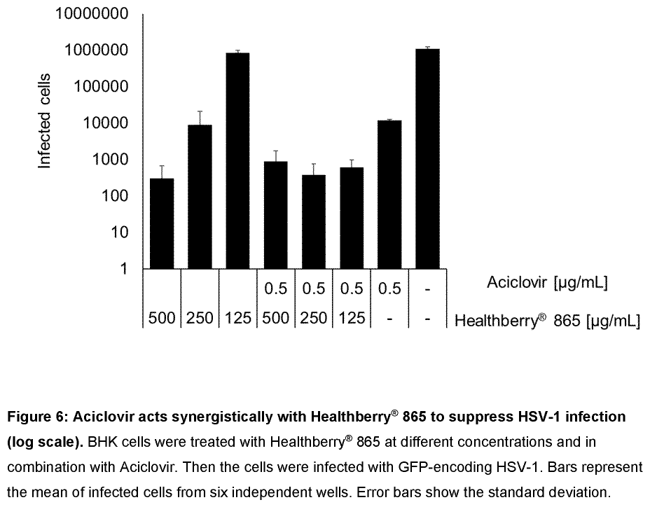

FIG. 6 shows that Aciclovir acts synergistically with Healthberry® 865 to suppress HSV-1 infection (log scale). BHK cells were treated with Healthberry® 865 at different concentrations as well as in combination with Aciclovir and then subsequently infected with GFP-encoding HSV-1.

### Example 7: Synergistic Effect of D3G and Aciclovir on Herpes Simplex Virus 1

Previous results have shown that D3G acts on the HSV-1 replication between virus entry and the early phase of virus replication since GFP-expression as readout is active in the early phase of viral gene expression. However, Aciclovir, the standard therapy for HSV-1, targets viral DNA replication before the late phase of virus replication cycle. Based on these aspects it was analyzed whether Aciclovir and D3G synergistically inhibit virus replication. BHK cells were incubated either with D3G alone or with D3G in combination with 2 μg/ml Aciclovir and subsequently infected with HSV-1. Two days after infection supernatants were collected, centrifuged to remove detached cells and used to infect BHK cells. After two addition days infected cells were quantified.

While D3G alone reduced viral infectivity about one order of magnitude the treatment with 2 μg/ml Aciclovir resulted in a reduction of two orders of magnitude. A combination of D3G and Aciclovir showed synergistic effects at higher doses of D3G (75 and 150 μg/mL) and even reduced viral infectivity in the range of three orders of magnitude (absolute values display a reduction from 1 million infected cells to 1 thousand). These experiments provided evidence that D3G is more effective on a complete replication cycle and that Aciclovir acts synergistically with D3G, indicating that patients might benefit from combination therapy.

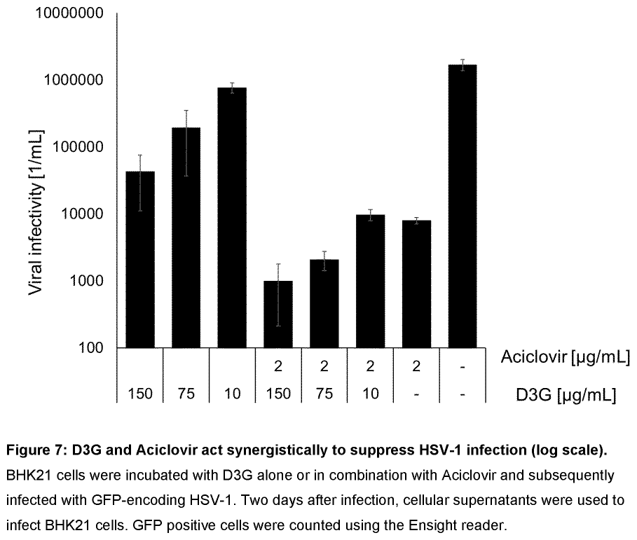

FIG. 7 shows that D3G and Aciclovir act synergistically to suppress HSV-1 infection (log scale). BHK cells were treated with D3G at different concentrations as well as in combination with Aciclovir and then subsequently infected with GFP-encoding HSV-1.

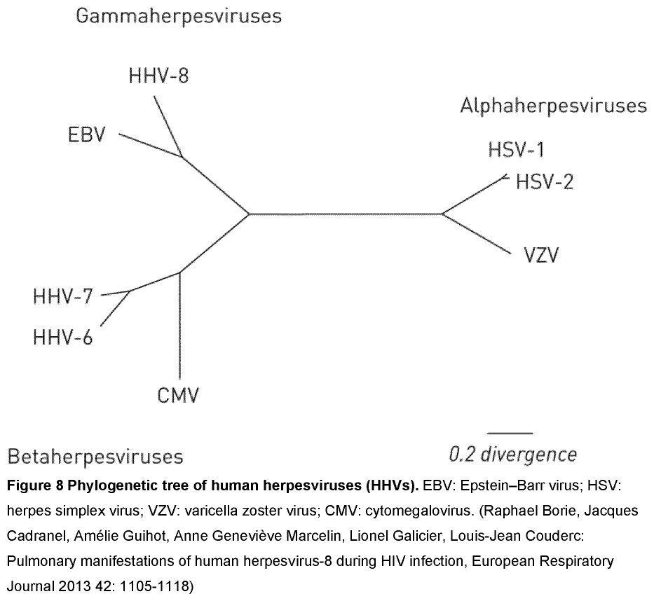

FIG. 8 shows the phylogenetic tree of human herpesviruses (HHVs). EBV: Epstein-Barr virus; HSV: herpes simplex virus; VZV: varicella zoster virus; CMV: cytomegalovirus. (Raphael Borie, Jacques Cadranel, Amélie Guihot, Anne Geneviéve Marcelin, Lionel Galicier, Louis-Jean Couderc: Pulmonary manifestations of human herpesvirus-8 during HIV infection, European Respiratory Journal 2013 42: 1105-1118). It is obvious from the phylogenetic tree that the human herpesviruses, which were tested, are located at different arms of the phylogenetic tree, covering members of the Gammaherpesviruses, Alphaherpesviruses and Betaherpesviruses. Therefore, it is to be expected that the antiviral activity of the berry extracts covers the whole family of Herpesviridae.

1. A combined preparation, comprising:
an anthocyanin composition; and
an antiviral agent suitable for treating or preventing a virus infection in a subject,
wherein the anthocyanin composition comprises an extract of black currants, an extract of bilberries, an extract of red grapes, and/or delphinidin 3 glucoside (D3G),
wherein the virus is from the Herpesviridae family and the antiviral agent is a Herpesviridae antiviral agent, and
wherein the anthocyanin composition and the antiviral agent are suitable for simultaneous, separate or sequential use with respect to each other.

2. The combined preparation of claim 1, wherein the anthocyanin composition comprises the extract of black currants and the extract of bilberries.

3. The combined preparation of claim 1, which is a composition comprising the anthocyanin composition and the antiviral agent.

4. A method of treating or preventing a virus infection in a subject, the method comprising:
administering an anthocyanin composition to the subject, either (i) in combination with an antiviral agent active against the virus or (ii) after the antiviral agent is administered to the subject,
wherein the anthocyanin composition comprises an extract of black currants, an extract of bilberries, an extract of red grapes, and/or delphinidin 3 glucoside, and
wherein the virus is from the Herpesviridae family and the antiviral agent is a Herpesviridae antiviral agent.

5. A method of treating or preventing a virus infection in a subject, the method comprising:
administering an antiviral agent to the subject, either (i) in combination with an anthocyanin composition comprising an extract of black currants, an extract of bilberries, an extract of red grapes, or delphinidin 3 glucoside active against the virus or (ii) after the anthocyanin composition is administered to the subject,
wherein the virus is from the Herpesviridae family and the antiviral agent is a Herpesviridae antiviral agent.

6. The combined preparation of claim 1, wherein the antiviral agent is an inhibitor of DNA replication, optionally wherein the antiviral agent is a DNA polymerase inhibitor or a DNA terminase complex inhibitor.

7. The combined preparation of claim 1, wherein the antiviral agent is a nucleoside analogue or a pyrophosphate analogue, or wherein the antiviral agent is a prodrug of a nucleoside analogue or a pyrophosphate analogue.

8. The combined preparation of claim 1, wherein the antiviral agent is acyclovir, ganciclovir, valganciclovir, foscarnet, famciclovir, penciclovir, valaciclovir, and/or letermovir.

9. The combined preparation of claim 1, wherein the anthocyanin composition comprises the extract of black currants, and the black currants are a fruit of Ribes nigrum, and/or
wherein the anthocyanin composition comprises the extract of bilberries, and the bilberries are a fruit of Vaccinium myrtillus.

10. The combined preparation of claim 1, wherein the extract of black currants, the extract of billberries, and/or the extract of red grapes is an extract of pomaces from the black currants, the bilberries and/or the red grapes, and is comprised by the anthocyanin preparation.

11. The combined preparation of claim 1, which comprises anthocyanins at a concentration of at least 25 weight-%.

12. The combined preparation of claim 1, wherein the extract of black currants, the extract of billberries, and/or the extract of red grapes is an alcoholic extract, and is comprised by the anthocyanin preparation.

13. The combined preparation of claim 1, wherein the extract of black currants, and/or the extract of billberries is comprised by the combined preparation, and is prepared by a process comprising:
extracting the black currants and/or the bilberries such that a product is obtained,
purifying the product via chromatography such that a purified product is obtained,
mixing the purified product with water such that a mixture is obtained, and
spray-drying the mixture.

14. The combined preparation of claim 1, which comprises cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, and/or delphinidin-3-rutinoside.

15. The combined preparation of claim 1, wherein the virus is herpes simplex virus-1 (HSV-1), herpes simplex virus-2 (HSV-2), Varicella zoster virus (VZV), Epstein-Barr virus (EBV), Cytomegalovirus (CMV), Roseolovirus, or Kaposi's sarcoma-associated herpesvirus (KSHV, HHV 8).

16. The combined preparation of claim 1, wherein: (i) the virus is Cytomegalovirus (CMV) and the antiviral agent is ganciclovir; or (ii) the virus is herpes simplex virus-1 (HSV-1) or Epstein-Barr virus (EBV) and the antiviral agent is acyclovir.

17. A method of treating or preventing a virus infection in a subject, the method comprising:
administering the combined preparation of claim 1 to the subject in 1 to 10 oral dosages of at least 80 mg anthocyanins each per day.

18. A method of treating or preventing a virus infection in a subject, the method comprising:
administering the combined preparation of claim 1 to the subject in 1 to 10 oral dosages of at least 20 mg D3G each per day.

19. A method of treating or preventing a virus infection in a subject, the method comprising:
administering the anthocyanin composition of the combined preparation of claim 1 to the subject such that the extract of black currants, the extract of billberries, and/or the extract of red grapes reaches a concentration in a target compartment of at least 30 μg/ml.

20. A method of treating or preventing a virus infection in a subject, the method comprising:
administering the combined preparation of claim 1 to the subject with a medical device,
optionally wherein the medical device is transdermal or endotracheal.

21. The method of claim 20, wherein the combined preparation is administered at a site of insertion of the medical device into the subject.

22. The method of claim 20, wherein the medical device is for provides endotracheal intubation or parenteral nutrition.

23. The method of claim 20, wherein the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter.

24. The method of claim 20, wherein a dwell time of the medical device in the subject is more than 24 hours.

25. A method of treating or preventing a virus infection in a subject, the method comprising:
administering the combined preparation of claim 1 to the subject,
wherein the subject is a human.

26. A method of treating or preventing a virus infection in a subject, the method comprising:
administering the combined preparation of claim 1 to the subject,
wherein the subject is a carrier of the virus.

27. A method of treating or preventing a virus infection in a subject, the method comprising:
administering the combined preparation of claim 1 to the subject,
wherein the subject is infected with Kaposi's sarcoma-associated herpesvirus (KSHV, HHV-8), and optionally is a carrier of Human immunodeficiency virus (HIV) or has Acquired immunodeficiency syndrome (AIDS).

28. The combined preparation of claim 1, wherein the virus infection is in a liver or kidney.

29. A method of treating or preventing, in a subject, a cancer associated with a virus from the Herpesviridae family, the method comprising:
administering the combined preparation of claim 1 to the subject, optionally wherein:
(i) the virus is Epstein-Barr virus (EBV) and the cancer is lymphoma (optionally Hodgkin lymphoma or Burkitts lymphoma), nasopharyngeal cancer, gastric cancer, or breast cancer; or
(ii) the virus is Kaposi's sarcoma-associated herpesvirus (KSHV, HHV-8) and the cancer is Kaposi's sarcoma, primary effusion lymphoma, HHV-8-associated multicentric Castleman disease, or breast cancer.

30. A method of treating or preventing, in a subject, an autoimmune disease associated with a virus from the Herpesviridae family, the method comprising:
administering the combined preparation of claim 1 to the subject, optionally wherein:
(i) the virus is Epstein-Barr virus (EBV) and the autoimmune disease is systemic lupus erythematosus (SLE), rheumatoid arthritis (RA), Sjögren's syndrome or multiple sclerosis; or
(ii) the virus is herpes simplex virus-1 (HSV-1) and the autoimmune disease is multiple sclerosis.

31. The method of claim 30, wherein the autoimmune disease is Alzheimer's disease.

32. The method of claim 31, which reduces β-amyloid plaque formation, optionally by reducing or preventing an active virus infection.

33. The method of claim 31, which reduces brain tissue inflammation.

34. A composition, comprising:
an antiviral agent, and
an anthocyanin composition,
wherein the anthocyanin composition comprises an extract of black currants, an extract of bilberries, an extract of red grape, and/or delphinidin 3 glucoside, and
wherein the antiviral agent is a Herpesviridae antiviral agent.

35. A kit, comprising, in separate containers: (i) an antiviral agent; and (ii) an anthocyanin composition,
wherein the anthocyanin composition comprises an extract of black currants, an extract of bilberries, an extract of red grape, and/or delphinidin 3 glucoside, and
wherein the antiviral agent is a Herpesviridae antiviral agent.

36. A medical device, suitable for insertion into a subject, the medical device comprising a coating composition on an exterior surface of the medical device,
wherein the coating composition comprises: (i) an antiviral agent; and (ii) an anthocyanin composition,
wherein the anthocyanin composition comprises an extract of black currants, an extract of bilberries, an extract of red grape, and/or delphinidin 3 glucoside, and
wherein the antiviral agent is a Herpesviridae antiviral agent.

37. The medical device of claim 36, wherein the medical device is a needle, a catheter, a port, an intubation device or tube, a nebulizer, an implant, a vascular access catheter, a brain microcatheter, a peripherally inserted central catheter, a chronic central venous catheter, an implanted port, an acute central venous catheter, a midline catheter, a short peripheral intravenous catheter, or a dialysis catheter.

38. The composition of claim 34, wherein the antiviral agent is a DNA polymerase inhibitor or a DNA terminase complex inhibitor.

39. The composition of claim 34, wherein the antiviral agent is a nucleoside analogue or a pyrophosphate analogue, or wherein the antiviral agent is a prodrug of a nucleoside analogue or a pyrophosphate analogue.

40. The composition of claim 34, wherein the antiviral agent is acyclovir, ganciclovir, valganciclovir, foscarnet, famciclovir, penciclovir, valaciclovir, and/or letermovir.

41. The composition of claim 34, wherein the anthocyanin composition comprises the extract of black currants and the extract of bilberries.

42. The composition of claim 34, which is a topical composition or eye drops.

43. The composition of claim 34, wherein the antiviral agent is acyclovir.

44. The composition of claim 34, wherein the antiviral agent is ganciclovir.

45. The composition of claim 34, which comprises a tonicity adjusting agent, a buffering agent, a preservative, an antioxidant, a stabilizer, a pH adjusting agent, a penetration enhancer, a surfactant and/or a humectant.

46. The composition of claim 34, which comprises an analgesic.

47. The composition of claim 34, comprising anthocyanins at a concentration of at least 25 weight-%.

48. The composition of claim 34, comprising at least 50 wt % extract.
